(19) United States
(12) Patent Application Publication
(10) Pub. No.: US 2023/0058911 A1
(43) Pub. Date: Feb. 23, 2023

(54) Urinary-Catheter Packages and Methods Thereof

(71) Applicant: C. R. Bard, Inc., Franklin Lakes, NJ (US)

(72) Inventors: Abigail Nabors, Longwood, FL (US); D. Claire Gloeckner, Lilburn, GA (US); Russell Riescher, Loganville, GA (US); Tom Roberts, Bishop, GA (US); Ana Minchew, Covington, GA (US)

(21) Appl. No.: 17/796,611

(22) PCT Filed: Dec. 30, 2020
(86) PCT No.: PCT/US20/67484

§ 371 Date: Jul. 29, 2022

Related U.S. Application Data
(60) Provisional application No. 62/968,040, filed on Jan. 30, 2020.

(51) Int. Cl. A61M 25/00; A61M 1/00

(52) U.S. Cl. CPC A61M 25/002; A61M 25/0017; A61M 1/84

(57) ABSTRACT

A urinary-catheter package (100) includes a container (120) including a urinary catheter (110) and a lubricant (140). The urinary catheter includes a catheter tube (112) and a drainage lumen extending through the urinary catheter, the drainage lumen fluidly coupling a plurality of eyelets in a distal-end portion of the catheter tube with an opening in a proximal end of the urinary catheter. The container includes a catheter section (122), a lubricant section (124) in a distal-end portion of the container, and a separator (126) between the catheter section and the lubricant section. The catheter section is configured to hold the urinary catheter, and the lubricant section is configured to hold the lubricant. The separator is configured to separate the lubricant in the lubricant section from the urinary catheter in the catheter section until the separator is broken by a user.

### PRIORITY

This application claims the benefit of priority to U.S. Provisional Application No. 62/968,040, filed Jan. 30, 2020, which is incorporated by reference in its entirety into this application.

### BACKGROUND

Users of urinary catheters such as intermittent catheters self-catheterize four to six times a day. An estimated 67% of such users in the United States purchase urinary catheters that are not pre-lubricated. These users carry tubes or packets of lubricant with them in order to lubricate their urinary catheters prior to catheterization. In efforts to speed up or simplify catheterization, the users might forego aseptic techniques during catheterization. For example, lubricant from a nonsterile multi-use lubricant container might be used, lubricant might be applied barehanded, or the like. Such techniques introduce a potential for contamination, which contamination, in turn, can lead to urinary tract infections (“UTIs”). While the users might believe occasional UTIs are normal and unavoidable, reducing the potential of contamination up front reduces potential systemic infections or other issues later. In addition, manually applying lubricant can result in uneven lubrication, which leads to friction and urethral irritation. In some cases, users might not even apply lubricant because they find the process messy or time-consuming.

Disclosed herein are urinary-catheter packages and methods thereof that address the foregoing.

### SUMMARY

Disclosed herein is a urinary-catheter package including, in some embodiments, a urinary catheter and a container including the urinary catheter and a lubricant for the urinary catheter. The urinary catheter includes a catheter tube and a drainage lumen extending through the urinary catheter. The drainage lumen fluidly couples a plurality of eyelets in a distal-end portion of the catheter tube with an opening in a proximal end of the urinary catheter. The container includes a catheter section, a lubricant section in a distal-end portion of the container, and a separator between the catheter section and the lubricant section. The catheter section is configured to hold the urinary catheter. The lubricant section is configured to hold the lubricant. The separator is configured to separate the lubricant in the lubricant section from the urinary catheter in the catheter section until the separator is broken by a user.

In some embodiments, the container includes a tapered outlet in the distal-end portion of the container. The tapered outlet is opposite the catheter section with the lubricant section therebetween.

In some embodiments, the container includes a first peelable seal over a major side of the tapered outlet. The first peelable seal is configured to create an opening of the tapered outlet when the first peelable seal is peeled away from the tapered outlet by the user in a proximal direction of the container.

In some embodiments, the first peelable seal includes a set of one or more indicators printed on the first peelable seal. The set of indicators is configured to guide the user in sizing the opening of the tapered outlet when peeling the first peelable seal from the tapered outlet.

In some embodiments, a size of the opening of the tapered outlet defines an amount of the lubricant that lubricates the catheter tube when the user distally advances the distal-end portion of the catheter tube from the catheter section of the container, through the lubricant section, and out the opening of the tapered outlet.

In some embodiments, the separator is a pierceable membrane configured to be pierced by a tip of the catheter tube when the user distally advances the distal-end portion of the catheter tube from the catheter section of the container into the lubricant section.

In some embodiments, the separator is a burstable membrane configured to be burst by pressure applied by the user to minor sides of the container.

In some embodiments, the separator is a pressed seal formed between major sides of the container. The pressed seal is coupled to a pull tab configured to unseal the pressed seal when the pull tab is pulled away from the pressed seal by the user.

In some embodiments, the container is configured to double as a handle when the distal-end portion of the catheter tube is disposed beyond the distal-end portion of the container. The handle allows the user to manipulate the urinary catheter without touching or contaminating the catheter tube.

In some embodiments, the container includes a second peelable seal over a proximal-end portion of the container. The second peelable seal is configured to create an opening of the proximal-end portion of the container when the second peelable seal is peeled away from the proximal-end portion of the container by the user in a distal direction of the container.

In some embodiments, the container is configured to double as a drainage extension tube for the urinary catheter when the distal-end portion of the catheter tube is disposed beyond the distal-end portion of the container and the second peelable seal is peeled away from the distal-end portion of the container.

Also disclosed herein is a urinary-catheter package including, in some embodiments, a urinary catheter, a package of lubricant for the urinary catheter, and a container including the urinary catheter and the package of lubricant. The urinary catheter includes a catheter tube, a funnel coupled to a proximal-end portion of the catheter tube, a plurality of eyelets in a distal-end portion of the catheter tube, and a drainage lumen extending through the urinary catheter. The drainage lumen fluidly couples the plurality of eyelets with an opening in a proximal end of the funnel. The package of lubricant is configured for easing insertion of the urinary catheter into a urethra of a user. The container includes a primary section configured to hold the urinary catheter and the package of lubricant. The container also includes a tapered outlet in a distal-end portion of the container. The package of lubricant is disposed in the primary section between the urinary catheter and the tapered outlet.

In some embodiments, the container includes a first peelable seal over a major side of the tapered outlet. The first peelable seal is configured to create an opening of the tapered outlet when the first peelable seal is peeled away from the tapered outlet by the user in a proximal direction of the container.

In some embodiments, the first peelable seal includes a set of one or more indicators printed on the first peelable seal. The set of indicators is configured to guide the user in sizing the opening of the tapered outlet when peeling the first peelable seal from the tapered outlet.

In some embodiments, a size of the opening of the tapered outlet defines an amount of the lubricant that lubricates the catheter tube when the user distally advances the distal-end portion of the catheter tube from the primary section of the container, through released lubricant, and out the opening of the tapered outlet.

In some embodiments, the package of lubricant is configured to be burst and release lubricant by pressure applied by the user to minor sides of the container around the package of lubricant.

In some embodiments, the container is configured to double as a handle when the distal-end portion of the catheter tube is disposed beyond the distal-end portion of the container. The handle allows the user to manipulate the urinary catheter without touching or contaminating the catheter tube.

In some embodiments, the container includes a second peelable seal over a proximal-end portion of the container. The second peelable seal is configured to create an opening of the proximal-end portion of the container when the second peelable seal is peeled away from the proximal-end portion of the container by the user in a distal direction of the container.

In some embodiments, the container is configured to double as a drainage extension tube for the urinary catheter when the distal-end portion of the catheter tube is disposed beyond the distal-end portion of the container and the second peelable seal is peeled away from the proximal-end portion of the container.

Also disclosed herein is a container for a urinary-catheter package including, in some embodiments, a catheter section, a lubricant section in a distal-end portion of the container, a separator between the catheter section and the lubricant section, and a tapered outlet in the distal-end portion of the container. The catheter section is configured to hold the urinary catheter. The lubricant section is configured to hold lubricant for lubricating at least a distal-end portion of a catheter tube of the urinary catheter. The separator is configured to separate the lubricant section from the catheter section until the separator is broken by a user. The tapered outlet is opposite the catheter section with the lubricant section therebetween.

In some embodiments, the container includes a first peelable seal over a major side of the tapered outlet. The first peelable seal is configured to create an opening of the tapered outlet when the first peelable seal is peeled away from the tapered outlet in a proximal direction of the container.

In some embodiments, the first peelable seal includes a set of one or more indicators printed on the first peelable seal. The set of indicators is configured to guide the user in sizing the opening of the tapered outlet when peeling the first peelable seal from the tapered outlet.

In some embodiments, a size of the opening of the tapered outlet defines an amount of the lubricant that lubricates the catheter tube when the user distally advances the distal-end portion of the catheter tube from the catheter section of the container, through the lubricant section, and out the opening of the tapered outlet.

In some embodiments, the separator is a pierceable membrane configured to be pierced by a tip of the catheter tube when the user distally advances the distal-end portion of the catheter tube from the catheter section of the container into the lubricant section.

In some embodiments, the separator is a burstable membrane configured to be burst by pressure applied by the user to minor sides of the container.

In some embodiments, the separator is a pressed seal formed between major sides of the container. The pressed seal is coupled to a pull tab configured to unseal the pressed seal when the pull tab is pulled away from the pressed seal by the user.

In some embodiments, the container is configured to double as a handle when the distal-end portion of the catheter tube is disposed beyond the distal-end portion of the container. The handle allows the user to manipulate the urinary catheter without touching or contaminating the catheter tube.

In some embodiments, the container includes a second peelable seal over a proximal-end portion of the container. The second peelable seal is configured to create an opening of the proximal-end portion of the container when the second peelable seal is peeled away from the proximal-end portion of the container by the user in a distal direction of the container.

In some embodiments, the container is configured to double as a drainage extension tube for the urinary catheter when the distal-end portion of the catheter tube is disposed beyond the distal-end portion of the container and the second peelable seal is peeled away from the proximal-end portion of the container.

Also disclosed herein is a method for a urinary-catheter package including, in some embodiments, an obtaining step of obtaining the urinary-catheter package. The urinary-catheter package includes a urinary catheter in a catheter section of a container and a lubricant in a lubricant section of the container. The method also includes a first peeling step of peeling a first peelable seal away from a tapered outlet in a distal-end portion of the container. The first peeling step creates an opening of the tapered outlet. The method also includes a breaking step of breaking a separator separating the lubricant in the lubricant section from the urinary catheter in the catheter section. The method also includes a first advancing step of advancing a distal-end portion of a catheter tube of the urinary catheter from the catheter section of the container, through the lubricant section, and out the opening of the tapered outlet.

In some embodiments, the first peeling step includes peeling the first peelable seal to an indicator of a set of one or more indicators printed on the first peelable seal. Thus, the first peeling step includes sizing the opening of the tapered outlet in accordance with the indicator during the first peeling step.

In some embodiments, sizing the opening of the tapered outlet defines an amount of the lubricant that lubricates the catheter tube during the first advancing step.

In some embodiments, the breaking step includes piercing a pierceable membrane by a tip of the catheter tube during the first advancing step.

In some embodiments, the breaking step includes bursting a burstable membrane by applying pressure to minor sides of the container before the first advancing step.

In some embodiments, the breaking step includes pulling a pull tab coupled to a pressed seal formed between major sides of the container away from the pressed seal before the first advancing step.

In some embodiments, the method further includes an inserting step of inserting the distal-end portion of the catheter tube into a urethral meatus. The method also includes a second advancing step of advancing a distal end of the catheter tube into a bladder until urine is observed.

In some embodiments, the method further includes a handling step of handling a proximal-end portion of the urinary catheter by the distal-end portion of the container during the second advancing step. The container doubles as a handle during the handling step, which allows a user to manipulate the urinary catheter without touching or contaminating the catheter tube.

In some embodiments, the method further includes a draining step of draining the urine through an opening in a proximal-end portion of the container. The container doubles as a drainage extension tube. The drainage extension tube is fluidly coupled to the bladder by way of a drainage lumen of the catheter tube that extends from a plurality of eyelets in the distal-end portion of the catheter tube to an opening in a proximal end of the urinary catheter.

In some embodiments, the method further includes a second peeling step of peeling a second peelable seal away from the proximal-end portion of the container. The second peeling step includes creating the opening of the proximal-end portion of the container.

These and other features of the concepts provided herein will become more apparent to those of skill in the art in view of the accompanying drawings and following description, which describe particular embodiments of such concepts in greater detail.

### DRAWINGS

FIG. 1 illustrates a perspective view urinary-catheter package in accordance with some embodiments.

FIG. 2 illustrates a detailed view of a proximal-end portion of the urinary-catheter package of FIG. 1.

FIG. 3 illustrates simplified view of the urinary-catheter package having a first alternative separator in accordance with some embodiments.

FIG. 4 illustrates simplified view of the urinary-catheter package having a second alternative separator in accordance with some embodiments.

FIG. 5 illustrates simplified view of an alternative urinary-catheter package in accordance with some embodiments.

FIG. 6 illustrates a container of the urinary-catheter package doubling as a drainage extension tube for a urinary catheter in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

### Urinary-Catheter Packages

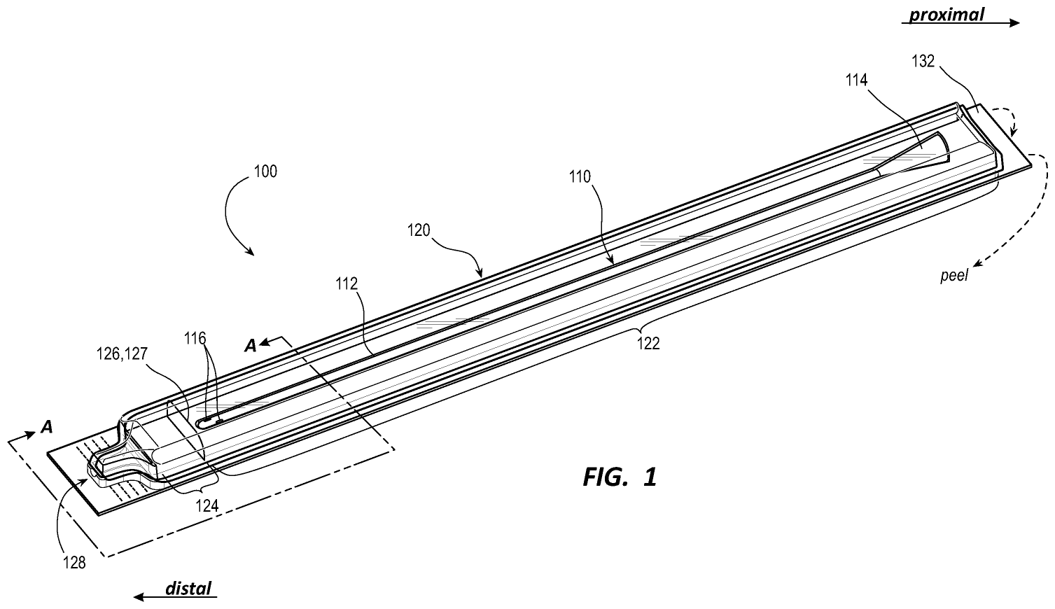

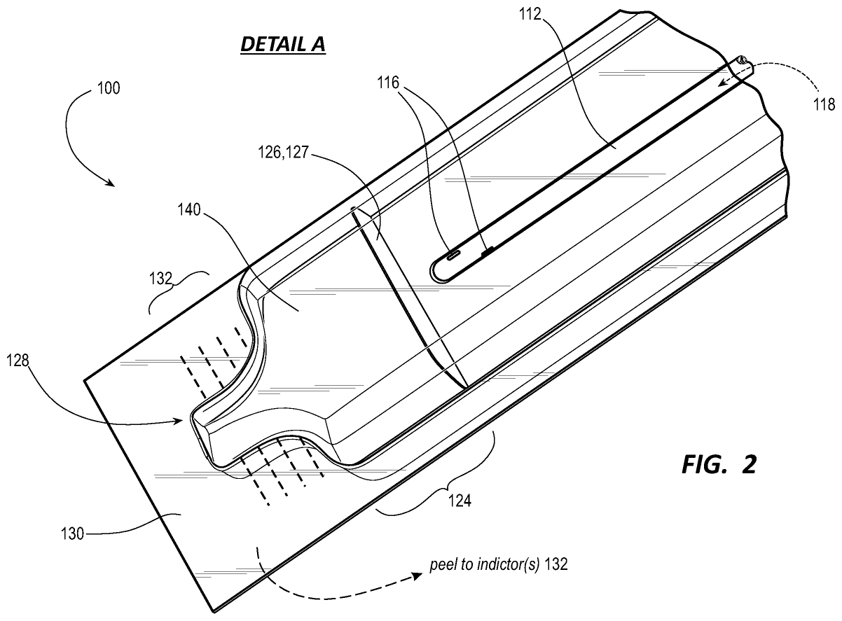

FIG. 1 illustrates a perspective view urinary-catheter package 100 in accordance with some embodiments. FIG. 2 illustrates a detailed view of a proximal-end portion of the urinary-catheter package 100 of FIG. 1.

As shown, the urinary-catheter package 100 includes a urinary catheter 110 and a container 120 including the urinary catheter 110 and a lubricant 140 for the urinary catheter, for example, for easing insertion of the urinary catheter 100 into a urethra of a user.

The urinary catheter 110 (e.g., an intermittent catheter) includes a catheter tube 112, a funnel 114 coupled to a proximal-end portion of the catheter tube 112, a plurality of eyelets 116 in a distal-end portion of the catheter tube 112, and a drainage lumen 118 extending through the urinary catheter 110. The drainage lumen 118 fluidly couples the plurality of eyelets 116 with an opening in a proximal end of the funnel 114. While the urinary catheter 110 can be coated with a lubricious coating or a coating that becomes lubricious in the presence of water vapor or liquid water, the urinary-catheter package 100 is designed to generally include the urinary catheter 110 without such a coating.

The container 120 (e.g., a form-fill-seal [“FFS”] pouch) includes a catheter section 122, a lubricant section 124 in a distal-end portion of the container 120, a separator 126 between the catheter section 122 and the lubricant section 124, a tapered outlet 128 in the distal-end portion of the container 120, a first peelable seal 130 over a major side of the tapered outlet 128, and a second peelable seal 132 over a proximal-end portion of the container.

The catheter section 122 is configured to hold the urinary catheter 110.

The lubricant section 124 is configured to hold the lubricant 140.

The separator 126 is configured to separate the lubricant 140 in the lubricant section 124 from the urinary catheter 100 in the catheter section 122 until the separator 126 is broken by the user.

As shown in FIGS. 1 and 2, the separator 126 can be a pierceable membrane 127 configured to be pierced by a tip in the distal-end portion of the catheter tube 112 when the user distally advances the distal-end portion of the catheter tube 112 from the catheter section 122 of the container 120 into the lubricant section 124.

First and second alternatives to the pierceable membrane 127 as the separator 126 are set forth below.

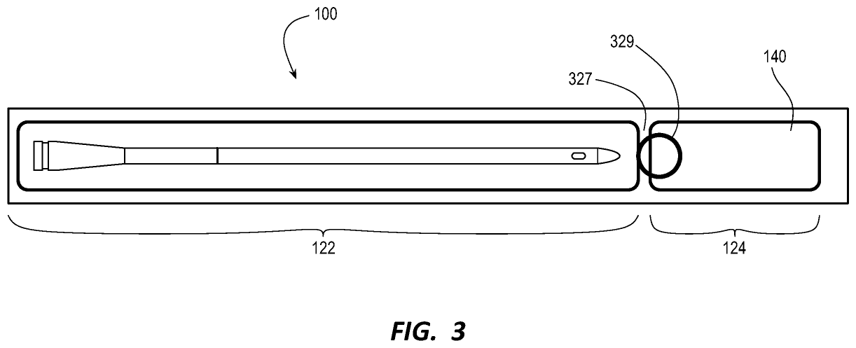

FIG. 3 illustrates simplified view of the urinary-catheter package 100 having a pressed seal 327 as the separator 126 in accordance with some embodiments.

As shown, the pressed seal 327 can be formed between major sides of the container 120; however, the pressed seal 327 can be formed between minor sides of the container 120 in other embodiments. The pressed seal 327 is coupled to at least one pull tab 329 configured to unseal the pressed seal 327 when the pull tab 329 is pulled away from the pressed seal 327 by the user. If the urinary-catheter package 100 includes two pull tabs, the two pull tabs can be on opposite sides (e.g., opposite major sides or opposite minor sides) of the container 120 to oppose each other when pulled.

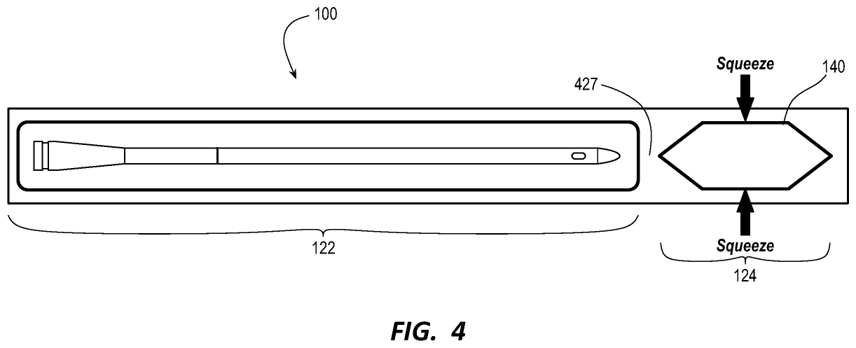

FIG. 4 illustrates simplified view of the urinary-catheter package 100 having a burstable membrane 427 as the separator 126 in accordance with some embodiments.

As shown, the burstable membrane 427 can be configured to be burst by pressure applied by the user (e.g., by squeezing between a thumb and one or more opposing fingers) on the minor sides of the container 120; however, the burstable membrane 427 can be configured to be burst by pressure applied by the user on the major sides of the container 120 in other embodiments. With respect to the embodiment shown in FIG. 4, the lubricant section 124 is configured to be relatively wide in a medial portion of the lubricant section 124 and relatively narrow in a proximal-end portion of the lubricant section 124 with a shoulder portion therebetween. The proximal-end portion of the lubricant section 124 can also have a relatively pointed proximal end corresponding to a thinner, weaker portion of the burstable membrane 427. Such dimensions for the lubricant section 124 direct a portion of the lubricant 140 into the distal-end section of the catheter section 122 of the container 120 when the burstable membrane 427 is burst by the user.

The tapered outlet 128 is opposite the catheter section 122 with the lubricant section 124 therebetween.

The first peelable seal 130 is configured to create an opening of the tapered outlet 128 when the first peelable seal 130 is peeled away from the tapered outlet 128 by the user in a proximal direction of the container.

The first peelable seal 130 includes a set of one or more indicators 132 printed on the first peelable seal 130. The set of indicators 132 is configured to guide the user in consistently sizing the opening of the tapered outlet 128 as desired when peeling the first peelable seal 130 from the tapered outlet 128. A size of the opening of the tapered outlet 128 defines an amount of the lubricant 140 that lubricates the catheter tube 112 when the user distally advances the distal-end portion of the catheter tube 112 from the catheter section 122 of the container 120, through the lubricant section 124, and out the opening of the tapered outlet 128.

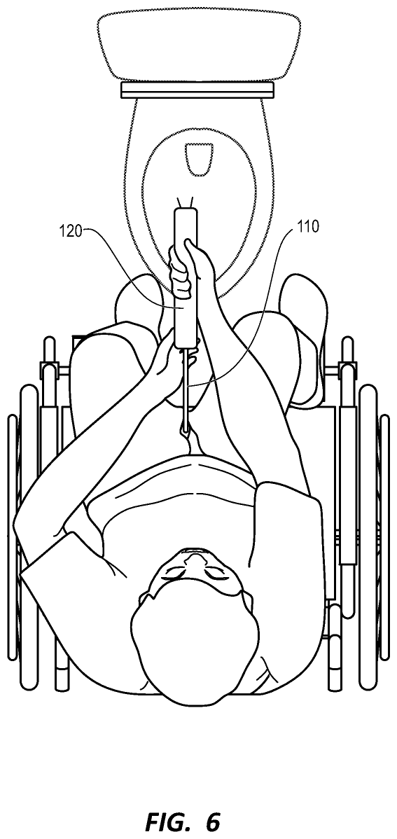

Advantageously, the container 120 is configured to double as a handle or grip when the distal-end portion of the catheter tube 112 is disposed beyond the distal-end portion or a distal end of the container 120. The handle allows the user to manipulate the urinary catheter 110 without touching or contaminating the catheter tube 112, thereby maintaining aseptic technique and minimizing the potential for UTIs. (See, for example, FIG. 6, which alludes to how the user can use the container 120 as a handle or grip.)

The container 120 includes a second peelable seal 132 over a proximal-end portion of the container 120. The second peelable seal 132 is configured to create an opening of the proximal-end portion of the container 120 when the second peelable seal 132 is peeled away from the proximal-end portion of the container 120 by the user in a distal direction of the container 120. When created, the opening of the proximal-end portion of the container 120 allows the user to use the container 120 as a drainage extension tube.

FIG. 6 illustrates the container 120 of a urinary-catheter package 100 doubling as a drainage extension tube for the urinary catheter 110 in accordance with some embodiments.

As shown, the container 120 is configured to double as a drainage extension tube for the urinary catheter 110 when the distal-end portion of the catheter tube 112 is disposed beyond the distal-end portion or the distal end of the container 120 and the second peelable seal 132 is peeled away from the distal-end portion of the container 120 to form the opening of the proximal-end portion of the container 120. Such a drainage extension tube can be useful to those whom experience difficulty getting close enough to a toilet or urinal to drain urine directly from the proximal end of the urinary catheter 110 or the funnel 114 thereof. Indeed, FIG. 6 illustrates how a person in a wheelchair might use the urinary-catheter package 100.

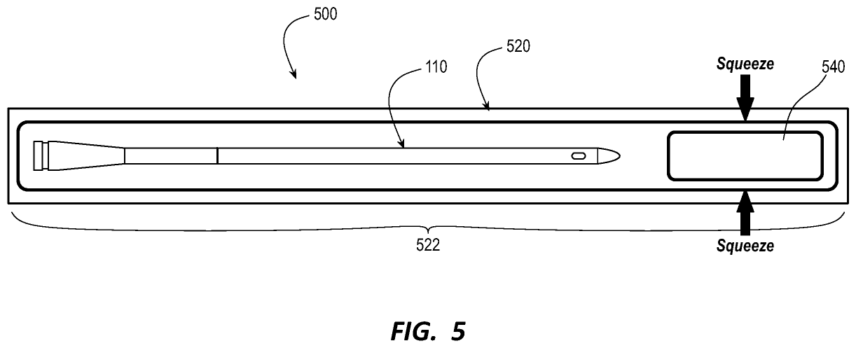

FIG. 5 illustrates simplified view of an alternative urinary-catheter package 500 in accordance with some embodiments.

As shown, the urinary-catheter package 500 includes the urinary catheter 110, a package of lubricant 540 for the urinary catheter 110, and a container 520 including the urinary catheter 110 and the package of lubricant 540.

The container 520 can be largely similar or even identical to the container 120 of the urinary-catheter package 100 except for the catheter section 122, the lubricant section 124, and the separator 126 between the catheter section 122 and the lubricant section 124. Instead of the foregoing sections and the separator 126 therebetween, the container 520 includes a primary section 522 configured to hold both the urinary catheter 110 and the package of lubricant 540. In the urinary-catheter package 500, the package of lubricant 540 is disposed in the primary section 522 between the urinary catheter 110 and the tapered outlet 128 so as to function as set forth above to lubricate the catheter tube 112 when the user distally advances the urinary catheter 110 out the tapered outlet 128 of the container 120.

Like the burstable membrane 427 of the container 120 that separates the lubricant 140 of the lubricant section 124 from urinary catheter 110 of the catheter section 122, the package of lubricant 540 is configured to be burst by pressure applied by the user (e.g., by squeezing between a thumb and one or more opposing fingers) on the major or minor sides of the container 520 to release the lubricant 140 from the package of lubricant 540. The package of lubricant 540 can be a thin-walled pouch or a soft-sided bottle with burstable membranes at its ends. The soft-sided bottle is advantageous in that it can function as a handle or grip when the catheter tube 112 is threaded therethrough.

It should be understood the package of lubricant 540 is distinct from the separator 126 or, in this case, the burstable membrane 427 in that the package of lubricant 540 is added to the container 520 with the urinary catheter 110 to form the urinary-catheter package 500. In contrast, the separator 126 is bodily incorporated into the container 120.

### Methods

Methods of the urinary-catheter package 100 or 500 include a method for using the urinary-catheter package 100.

The method for using the urinary-catheter package 100 include an obtaining step of obtaining the urinary-catheter package 100. As set forth above, the urinary-catheter package 100 includes the urinary catheter 110 in the catheter section 122 of the container 120 and the lubricant 140 in the lubricant section 124 of the container 120.

The method also includes a first peeling step of peeling (e.g., in a proximal direction of the container 120) the first peelable seal 130 away from the tapered outlet 128 in the distal-end portion of the container 120 to create an opening of the tapered outlet 128. The first peeling step includes peeling the first peelable seal 130 to an indicator of the set of one or more indicators 132 printed on the first peelable seal 130. Thus, the first peeling step includes sizing the opening of the tapered outlet 128 in accordance with the indicator during the first peeling step. Sizing the opening of the tapered outlet 128 defines an amount of the lubricant 140 that lubricates the catheter tube 112 during the first advancing step set forth below.

The method also includes a breaking step of breaking the separator 126 separating the lubricant 140 in the lubricant section 124 from the urinary catheter 110 in the catheter section 122. Because the separator 126 can be the pierceable membrane 127, the pressed seal 327, or the burstable membrane 427, the breaking step can vary. For example, the breaking step can include piercing the pierceable membrane 127 by the tip of the catheter tube 112 during the first advancing step set forth below. In another example, the breaking step can include pulling the pull tab 329 coupled to the pressed seal 327 formed between the major or minor sides of the container 120 away from the pressed seal 327 before the first advancing step set forth below. In yet another example, the breaking step can include bursting the burstable membrane 427 by applying pressure to the major or minor sides of the container 120 before the first advancing step set forth below.

The method also includes a first advancing step of advancing the distal-end portion of the catheter tube 112 of the urinary catheter 110 from the catheter section 122 of the container 120, through the lubricant section 124, and out the opening of the tapered outlet 128.

The method can further include an inserting step of inserting the distal-end portion of the catheter tube 112 into a urethral meatus of the user.

The method can further include a second advancing step of advancing the distal end of the catheter tube 112 into a bladder of the user until urine is observed.

The method can further include a handling step of handling the proximal-end portion of the urinary catheter 110 by the distal-end portion of the container 120 during the second advancing step. As set forth above, the container 120 doubles as a handle or grip during the handling step, which allows the user to manipulate the urinary catheter 110 without touching or contaminating the catheter tube 112 thereof. The handling step maintains aseptic technique and minimizes the potential for UTIs. (See, for example, FIG. 6, which alludes to how the user can use the container 120 during the handling step.)

The method can further include a second peeling step of peeling (e.g., in a distal direction of the container 120) the second peelable seal 132 away from the proximal-end portion of the container 120. The second peeling step includes creating the opening of the proximal-end portion of the container 120.

The method can further include a draining step of draining the urine through the opening in the proximal-end portion of the container 120 using the container 120 as a drainage extension tube as set forth above. (See FIG. 6, which illustrates the draining step with the container 120 as the drainage extension tube.) The drainage extension tube is fluidly coupled to the bladder by way of the drainage lumen of the catheter tube 112, which drainage lumen extends from the plurality of eyelets 116 in the distal-end portion of the catheter tube 112 to the opening in the proximal end of the urinary catheter 110 or the funnel 114 thereof.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary-catheter package, comprising:
a urinary catheter including:
a catheter tube; and
a drainage lumen extending through the urinary catheter fluidly coupling a plurality of eyelets in a distal-end portion of the catheter tube with an opening in a proximal end of the urinary catheter; and
a container including the urinary catheter and a lubricant therefor, the container including:
a catheter section configured to hold the urinary catheter;
a lubricant section in a distal-end portion of the container configured to hold the lubricant for the urinary catheter;
a separator configured to separate the lubricant in the lubricant section from the urinary catheter in the catheter section until the separator is broken by a user.

2. The urinary-catheter package of claim 1, the container further including a tapered outlet in the distal-end portion of the container, the tapered outlet opposite the catheter section with the lubricant section therebetween.

3. The urinary-catheter package of claim 2, the container further including a first peelable seal over a major side of the tapered outlet, the first peelable seal configured to create an opening of the tapered outlet when the first peelable seal is peeled away from the tapered outlet by the user in a proximal direction of the container.

4. The urinary-catheter package of claim 3, wherein the first peelable seal includes a set of one or more indicators printed thereon, the set of indicators configured to guide the user in sizing the opening of the tapered outlet when peeling the first peelable seal from the tapered outlet.

5. The urinary-catheter package of claim 4, wherein a size of the opening of the tapered outlet defines an amount of the lubricant that lubricates the catheter tube when the user distally advances the distal-end portion of the catheter tube from the catheter section of the container, through the lubricant section, and out the opening of the tapered outlet.

6. The urinary-catheter package of claim 1, wherein the separator is a pierceable membrane configured to be pierced by a tip of the catheter tube when the user distally advances the distal-end portion of the catheter tube from the catheter section of the container into the lubricant section.

7. The urinary-catheter package of claim 1, wherein the separator is a burstable membrane configured to be burst by pressure applied by the user to minor sides of the container.

8. The urinary-catheter package of claim 1, wherein the separator is a pressed seal formed between major sides of the container, the pressed seal coupled to a pull tab configured to unseal the pressed seal when the pull tab is pulled away from the pressed seal by the user.

9. The urinary-catheter package of claim 1, wherein the container is configured to double as a handle when the distal-end portion of the catheter tube is disposed beyond the distal-end portion of the container, the handle allowing the user to manipulate the urinary catheter without touching or contaminating the catheter tube.

10. The urinary-catheter package of claim 1, the container further including a second peelable seal over a proximal-end portion of the container, the second peelable seal configured to create an opening of the proximal-end portion of the container when the second peelable seal is peeled away from the proximal-end portion of the container by the user in a distal direction of the container.

11. The urinary-catheter package of claim 10, wherein the container is configured to double as a drainage extension tube for the urinary catheter when the distal-end portion of the catheter tube is disposed beyond the distal-end portion of the container and the second peelable seal is peeled away from the distal-end portion of the container.

12. A urinary-catheter package, comprising:
a urinary catheter including:
a catheter tube;
a funnel coupled to a proximal-end portion of the catheter tube;
a plurality of eyelets in a distal-end portion of the catheter tube; and
a drainage lumen extending through the urinary catheter fluidly coupling the plurality of eyelets with an opening in a proximal end of the funnel; and
a container including the urinary catheter and a package of lubricant configured for easing insertion of the urinary catheter into a urethra of a user, the container including:
a primary section configured to hold the urinary catheter and the package of lubricant; and
a tapered outlet in a distal-end portion of the container, the package of lubricant disposed in the primary section between the urinary catheter and the tapered outlet.

13. The urinary-catheter package of claim 12, the container further including a first peelable seal over a major side of the tapered outlet, the first peelable seal configured to create an opening of the tapered outlet when the first peelable seal is peeled away from the tapered outlet by the user in a proximal direction of the container.

14. The urinary-catheter package of claim 13, wherein the first peelable seal includes a set of one or more indicators printed thereon, the set of indicators configured to guide the user in sizing the opening of the tapered outlet when peeling the first peelable seal from the tapered outlet.

15. The urinary-catheter package of claim 14, wherein a size of the opening of the tapered outlet defines an amount of the lubricant that lubricates the catheter tube when the user distally advances the distal-end portion of the catheter tube from the primary section of the container, through released lubricant, and out the opening of the tapered outlet.

16. The urinary-catheter package of claim 12, wherein the package of lubricant is configured to be burst and release lubricant by pressure applied by the user to minor sides of the container around the package of lubricant.

17. The urinary-catheter package of claim 12, wherein the container is configured to double as a handle when the distal-end portion of the catheter tube is disposed beyond the distal-end portion of the container, the handle allowing the user to manipulate the urinary catheter without touching or contaminating the catheter tube.

18. The urinary-catheter package of claim 12, the container further including a second peelable seal over a proximal-end portion of the container, the second peelable seal configured to create an opening of the proximal-end portion of the container when the second peelable seal is peeled away from the proximal-end portion of the container by the user in a distal direction of the container.

19. The urinary-catheter package of claim 18, wherein the container is configured to double as a drainage extension tube for the urinary catheter when the distal-end portion of the catheter tube is disposed beyond the distal-end portion of the container and the second peelable seal is peeled away from the proximal-end portion of the container.

20. A container for a urinary-catheter package, comprising:
a catheter section configured to hold a urinary catheter;
a lubricant section in a distal-end portion of the container configured to hold lubricant for lubricating at least a distal-end portion of a catheter tube of the urinary catheter;
a separator configured to separate the lubricant section from the catheter section until the separator is broken by a user; and
a tapered outlet in the distal-end portion of the container, the tapered outlet opposite the catheter section with the lubricant section therebetween.

21. The container of claim 20, the container further including a first peelable seal over a major side of the tapered outlet, the first peelable seal configured to create an opening of the tapered outlet when the first peelable seal is peeled away from the tapered outlet in a proximal direction of the container.

22. The container of claim 21, wherein the first peelable seal includes a set of one or more indicators printed thereon, the set of indicators configured to guide the user in sizing the opening of the tapered outlet when peeling the first peelable seal from the tapered outlet.

23. The container of claim 22, wherein a size of the opening of the tapered outlet defines an amount of the lubricant that lubricates the catheter tube when the user distally advances the distal-end portion of the catheter tube from the catheter section of the container, through the lubricant section, and out the opening of the tapered outlet.

24. The container of claim 20, wherein the separator is a pierceable membrane configured to be pierced by a tip of the catheter tube when the user distally advances the distal-end portion of the catheter tube from the catheter section of the container into the lubricant section.

25. The container of claim 20, wherein the separator is a burstable membrane configured to be burst by pressure applied by the user to minor sides of the container.

26. The container of claim 20, wherein the separator is a pressed seal formed between major sides of the container, the pressed seal coupled to a pull tab configured to unseal the pressed seal when the pull tab is pulled away from the pressed seal by the user.

27. The container of claim 20, wherein the container is configured to double as a handle when the distal-end portion of the catheter tube is disposed beyond the distal-end portion of the container, the handle allowing the user to manipulate the urinary catheter without touching or contaminating the catheter tube.

28. The container of claim 20, the container further including a second peelable seal over a proximal-end portion of the container, the second peelable seal configured to create an opening of the proximal-end portion of the container when the second peelable seal is peeled away from the proximal-end portion of the container by the user in a distal direction of the container.

29. The container of claim 28, wherein the container is configured to double as a drainage extension tube for the urinary catheter when the distal-end portion of the catheter tube is disposed beyond the distal-end portion of the container and the second peelable seal is peeled away from the proximal-end portion of the container.

30-39. (canceled)
